(19) United States
(12) Patent Application Publication
(10) Pub. No.: US 2020/0265947 A1
(43) Pub. Date: Aug. 20, 2020

(54) CLINICAL TEST SUPPORT SYSTEM, NON-TRANSITORY MACHINE-READABLE STORAGE MEDIUM IN WHICH CLINICAL TEST SUPPORT PROGRAM IS STORED, AND CLINICAL TEST SUPPORT METHOD

(71) Applicant: Hitachi, Ltd., Tokyo (JP)

(72) Inventors: Haruhiko Nishiyama, Tokyo (JP); Kunihiko Kido, Tokyo (JP); Toru Hisamitsu, Tokyo (JP)

(73) Assignee: Hitachi, Ltd., Tokyo (JP)

(21) Appl. No.: 16/648,304

(22) PCT Filed: Feb. 20, 2019
(86) PCT No.: PCT/JP2019/006417

§ 371 Date: Mar. 18, 2020

(30) Foreign Application Priority Data
May 29, 2018 (JP) 2018-102103

(51) Int. Cl. G16H 40/20; G06Q 10/06; G06Q 10/10; G16H 10/20

(52) U.S. Cl. CPC G16H 40/20; G06Q 10/0635; G06Q 10/10; G06F 7/57; G06Q 10/06395; G06Q 10/063118; G16H 10/20; G06Q 10/06375

(57) ABSTRACT

It is provided a clinical test support system, comprising an arithmetic unit and a storage device coupled to the arithmetic unit, and a visit plan preparation unit to prepare a visit plan for the execution facility of a clinical development monitor; and a prediction unit to calculate the evaluation index of the visit plan. The storage device stores evaluation index information including the record of information for the evaluation of the visit plan and a risk evaluation model including the record of the risk evaluation result of each execution facility. The prediction unit calculates an evaluation index used to execute the visit plan prepared by the visit plan preparation unit by referring to the evaluation index information and the risk evaluation model; and output a visit plan selected based on the evaluation index.

### CLAIM OF PRIORITY

The present application claims priority from Japanese patent application JP 2018-102103 filed on May 29, 2018, the content of which is hereby incorporated by reference into this application.

### BACKGROUND OF THE INVENTION

The present invention relates to a clinical test support system.

In a clinical test or a new drug clinical test (hereinafter collectively referred to as a clinical test), a clinical test requester (a drug manufacturer or a Contract Research Organization (CRO)) performs, for the purpose of detecting the occurrence of an adverse event and/or a missed numerical value requiring an inspection for example (hereinafter referred to as an incident) in a medical institution (hereinafter referred to as an execution facility) in which the clinical test is performed, an on-site monitoring in which a Clinical Research Associate (CRA) visits an execution facility to confirm data on-site and a central monitoring in which data is collected from a plurality of execution facilities to confirm the data.

Any incident found by the CRA during the visit causes a processing cost depending on the incident.

The background art of this technical field includes the following prior art. JP 2009-64200 A discloses a clinical test management device according to which a drug manufacturer consigns a clinical test to an outside clinical test specialized institution by accessing the clinical test management device using a drug manufacturer terminal device to request a clinical test to extract a clinical test person who can handle the clinical test from a clinical test person list stored in a clinical test person list DB. Then, the drug manufacturer inquires, via a mobile phone of the extracted clinical test person, whether the clinical test person is available for the clinical test in the clinical test specialized institution to determine and extract, from a clinical test coordinator list stored in the CRCDB, an appropriate clinical test coordinator based on the abilities and skills. Then, the drug manufacturer sends, to the mobile phone owned by the extracted clinical test coordinator, data for an instruction for the dispatch to the clinical test specialized institution. Upon receiving the instruction, the clinical test coordinator is dispatched to the clinical test specialized institution based on a set schedule to support the clinical test of the clinical test person. After the completion of the clinical test, the clinical test coordinator sends a clinical report to the drug manufacturer from which the request was made.

### SUMMARY OF THE INVENTION

The on-site monitoring of the execution facilities is performed by providing a regular visit to the respective execution facilities based on a predetermined visit plan. When a certain execution facility frequently has incidents or frequently has incidents requiring a higher handling cost than in other execution facilities, the visit plan was modified so that this execution facility can receive more-frequent on-site monitoring.

This modification is performed empirically and is not determined quantitively depending on the frequencies or types of the incidents. This modification does not include a concept of quantitively arranging the visit plan in consideration not only of the frequency of the visit to an execution facility frequently having incidents but also of an execution facility infrequently having incidents by considering the entire picture. The visit plan has a different evaluation index depending on a management index, thus failing to provide the consideration of the presentation of such an index that can provide the comparison among visit plans for the quality. For example, an insufficient consideration is provided on the cost frequently used as an evaluation index.

Furthermore, according to JP2009-64200A, although a Clinical Research Coordinator (CRC) having an appropriate level depending on the execution facility is selected, no consideration is paid on the cost.

The representative one of inventions disclosed in this application is outlined as follows. There is provided a clinical test support system, comprising: an arithmetic unit for performing a predetermined processing; and a storage device coupled to the arithmetic unit; a visit plan preparation unit in which the arithmetic unit is configured to prepare a visit plan for the execution facility of a clinical development monitor; and a prediction unit in which the arithmetic unit is configured to calculate the evaluation index of the visit plan, wherein the storage device stores evaluation index information including the record of information for the evaluation of the visit plan and a risk evaluation model including the record of the risk evaluation result of each execution facility, the prediction unit is configured to: calculate an evaluation index used to execute the visit plan prepared by the visit plan preparation unit by referring to the evaluation index information and the risk evaluation model; and output a visit plan selected based on the evaluation index.

According to one embodiment of the present invention, the CRA visit plan can be evaluated based on a predetermined evaluation index. The problem, configuration, and effect other than the above-described ones will be clarified through the following description of embodiments.

### BRIEF DESCRIPTION OF THE DRAWINGS

FIG. 1 is a diagram for illustrating a configuration of the clinical test support system according to an embodiment of the present invention.

FIG. 2 is a block diagram for illustrating the physical configuration of the clinical test support system 1 of this embodiment.

FIG. 3 is a schematic diagram for illustrating an example of a visit plan.

FIG. 4 is a diagram for illustrating an example of a configuration of an incident handling cost table.

FIG. 5 is a diagram for illustrating an example of a configuration of a risk evaluation model.

FIG. 6 is a diagram for illustrating an example of a configuration of a monitor unit price table.

FIG. 7 is a flowchart of a clinical test support procedure.

FIG. 8 is a diagram for illustrating a method of a cost simulation by a cost prediction unit.

FIG. 9 is a diagram for illustrating a configuration of a periphery of the risk evaluation model calculation unit.

FIG. 10 is a diagram for illustrating a configuration example of a risk evaluation parameter initial value table.

FIG. 11 is a diagram for illustrating a configuration example of an incident database.

FIG. 12 is a diagram for illustrating a configuration example of the test evaluation result database.

FIG. 13 is a flowchart of a procedure to calculate the risk evaluation model initial value.

### DETAILED DESCRIPTION OF THE PREFERRED EMBODIMENTS

The following section will describe an embodiment in which a new drug clinical test is supported by a clinical test support system 1 according to the embodiment of the present invention. However, the clinical test support system 1 of this embodiment also can be widely applied to general clinical tests in addition to clinical tests. In this embodiment, a cost will be described as an example of the evaluation index. However, various evaluation indexes can be used as described later.

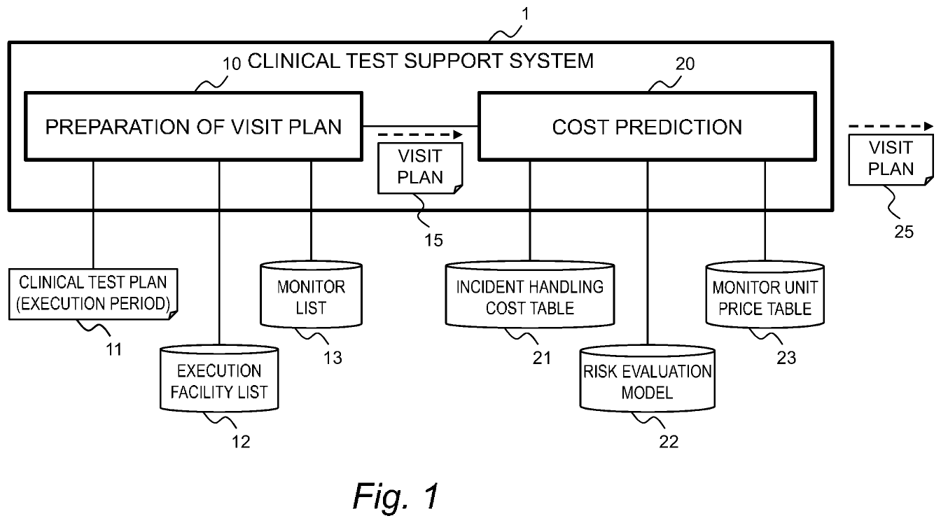

FIG. 1 is a diagram for illustrating a configuration of the clinical test support system 1 according to the embodiment of the present invention.

First, the following section will describe the outline of the function of the clinical test support system 1 of the embodiment of the present invention. The clinical test support system 1 of the embodiment configures a risk evaluation model 22 to estimate the incident occurrence frequency of each execution facility. The visit cost to be caused by the CRA is calculated by a cost prediction unit 20 using this risk evaluation model 22, a visit plan 15 for each execution facility, an incident handling cost table 21, and a monitor unit price table 23 for recording the cost of the Clinical Research Associate (CRA).

The cost prediction unit 20 selects a visit plan 25 for which the visit cost as an index to evaluate the visit plan is low. Based on the visit plan 25, a combination of the visit frequency of each execution facility and the CRA to visit the facility is determined. Based on this visit plan, the clinical test is monitored.

Prior to the start of a new clinical test for an execution facility, the risk evaluation model 22 for the execution facility is configured based on an incident database 33 for recording the past incident and the result of a test taken by the Clinical Research Coordinator (CRC) for the execution facility prior to the clinical test. The result of the test taken by the CRC is recorded by a test evaluation result database 34.

After the start of the clinical test, the incident information is collected as a monitoring record and the incident database 33 is updated. Thereafter, the risk evaluation model 22 is updated. Based on the updated risk evaluation model 22, the visit plan is reconsidered.

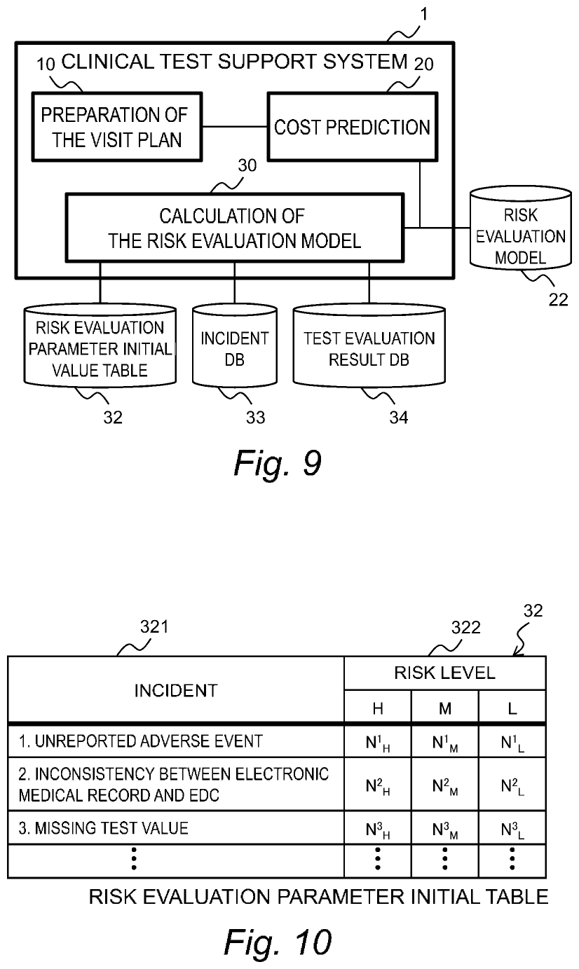

Next, with reference to FIG. 1, the following section will describe a configuration of the clinical test support system 1 of this embodiment. The clinical test support system 1 of this embodiment has a visit plan preparation unit 10 and the cost prediction unit 20. As described later with reference to FIG. 9, the clinical test support system 1 may optionally have a risk evaluation model calculation unit 30.

The visit plan preparation unit 10 prepares the visit plan 15 based on a clinical test plan 11, an execution facility list 12, and a monitor list 13. The visit plan 15 is a pattern based on which the CRA visits the execution facility at a predetermined timing (e.g., from an everyday visit to a one-time visit during a clinical test period), an example of which is shown in FIG. 3. The visit plan preparation unit 10 prepares many visit plans 15 for the cost prediction by the cost prediction unit 20 based on a timing at which the CRA visits the facility and/or a combination of the CRA and the execution facility. The clinical test plan 11 stores information for the to-be-executed clinical test (e.g., the execution period). The execution facility list 12 stores information for the execution facility planned to implement the clinical test. The monitor list 13 stores information for the CRA responsible for the clinical test.

Based on the incident handling cost table 21, the risk evaluation model 22, and the monitor unit price table 23, the cost prediction unit 20 calculates the costs of the respective visit plans 15 to output a visit plan 25 having a low cost.

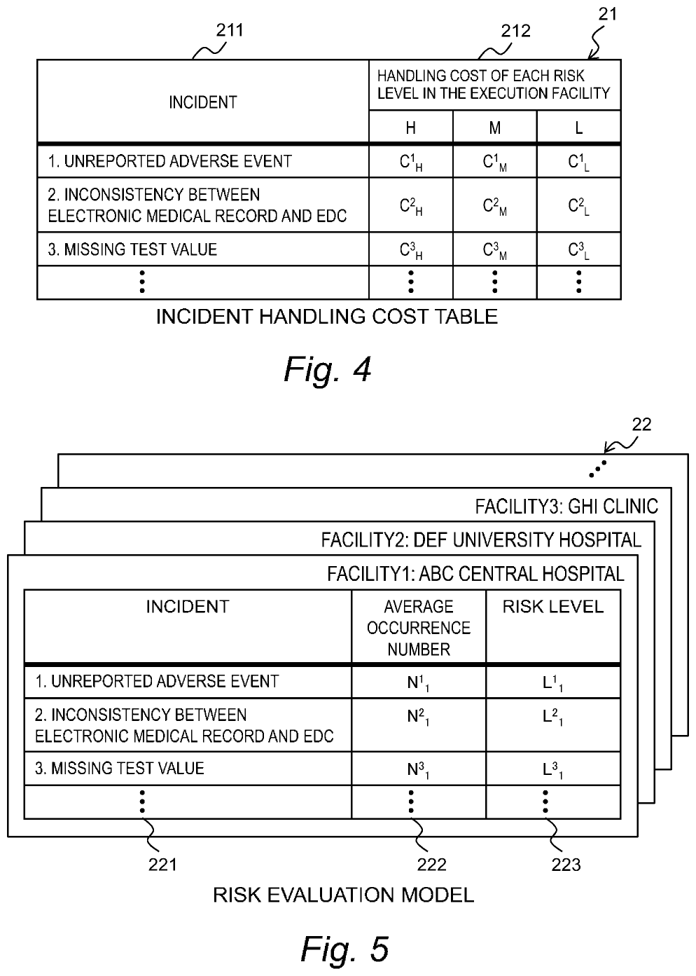

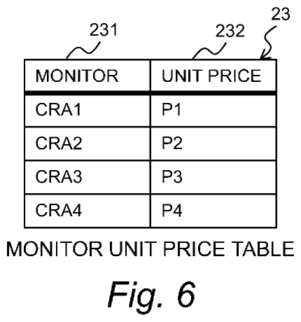

The incident handling cost table 21 records the costs required to handle the respective incidents occurred in clinical tests, a configuration example of which is shown in FIG. 4. The risk evaluation model 22 records the risk evaluation results of the respective execution facilities, a configuration example of which is shown in FIG. 5. A monitor unit price table 23 records the CRA costs, a configuration example of which is shown in FIG. 6.

The risk evaluation model calculation unit 30 generates the risk evaluation model 22 depending on the risk level of an execution facility, the details of which will be described later.

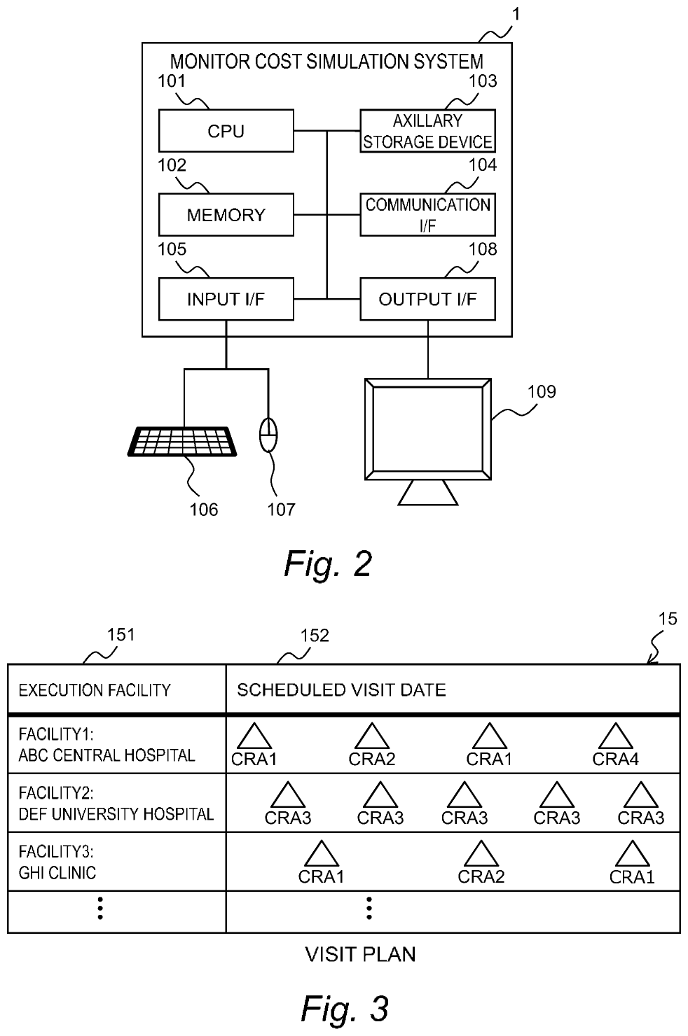

FIG. 2 is a block diagram for illustrating the physical configuration of the clinical test support system 1 according to this embodiment.

The clinical test support system 1 of this embodiment is configured by a computer having a processor (CPU) 101, a memory 102, an axillary storage device 103, a communication interface 104, an input interface 105, and an output interface 108.

The processor 101 is an arithmetic unit that executes a program stored in the memory 102. Programs executed by the processor 101 realize the functions of the clinical test support system 1. A program executed by the processor 101 may be partially executed by another arithmetic unit (e.g., FPGA).

The memory 102 includes an ROM as a nonvolatile storage element and a RAM as a volatile storage element. The ROM stores therein an unchangeable program (e.g., BIOS) for example. The RAM is a high-speed and volatile storage element such as a DRAM (Dynamic Random Access Memory) and temporarily stores therein a program to be executed by the processor 101 and data used for the execution of the program.

The axillary storage device 103 is a high-capacity and nonvolatile storage device such as a magnetic storage device (HDD) or a flash memory (SSD) for example. The axillary storage device 103 stores data used for the execution of a program by the processor 101 (e.g., the clinical test plan 11, the execution facility list 12, the monitor list 13, the visit plan 15, the incident handling cost table 21, the risk evaluation model 22, the monitor unit price table 23), and the program executed by the processor 101. Specifically, the program is read from the axillary storage device 103 and is loaded to the memory 102 and is executed by the processor 101 to thereby execute the respective functions of the clinical test support system 1.

The communication interface 104 is a network interface apparatus that controls the communication with other apparatuses based on a predetermined protocol.

The input interface 105 is an interface that is coupled to an input apparatus such as a keyboard 106 or a mouse 107 and that receives an input from an operator. The output interface 108 is an interface that is coupled to an output apparatus such as a display apparatus 109 or a printer (not shown) and that outputs the program execution result in a manner so that the result can be visually confirmed by an operator. Another configuration may be used in which the input apparatus and the output apparatus are provided by a terminal coupled to the clinical test support system 1 via a network.

A program executed by the processor 101 is provided via a removable media (e.g., CD-ROM, flash memory) or a network to the clinical test support system 1 and is stored in the nonvolatile axillary storage device 103 as a non-temporal storage medium. Thus, the clinical test support system 1 may have an interface to read data from removable media.

The clinical test support system 1 is a computer system physically configured on one computer or on a plurality of logically or physically configured computers. The clinical test support system 1 also may operate on a virtual computer configured on a plurality of physical computer resources.

FIG. 3 is a schematic diagram for illustrating an example of the visit plan 15. In an actual case, the visit plan 15 is recorded as a database in a data format such as a table for example.

The visit plan 15 records CRA visit schedules for the respective execution facilities and includes an execution facility 151 and a scheduled visit date 152. The visit plan 15 is prepared by the visit plan preparation unit 10 and is used as a parameter for a cost simulation by the cost prediction unit 20.

The execution facility 151 is a medical institution in which a clinical test is performed for example. The scheduled visit date 152 is a date on which the CRA is planned to visit the execution facility and is recorded together with information by which the CRA to visit the facility can be identified (e.g., ID, name).

FIG. 4 is a diagram for illustrating an example of a configuration of the incident handling cost table 21.

The incident handling cost table 21 includes the records of the costs required to handle the respective incidents occurred in clinical tests and includes an incident 211 and a handling cost 212.

The incident 211 shows the type of an incident occurring in a clinical test and is recorded to include the name and code of the incident as an incident type. The handling costs 212 are classified based on the risk levels of execution facilities (e.g., three risk stages of H: high risk, M: medium risk, L: low risk) so that the handling cost of one incident for each risk level is recorded as a positive value represented by a currency unit for example. The risk level of the execution facility is not limited to the illustrated three risk stages and may be represented by any number of stages. The handling cost of each incident may be calculated in advance by a specialist.

In the case of the occurrence of an unreported adverse event recorded in the incident handling cost table 21 illustrated in FIG. 4, then such a cost occurs that is required to have a hearing for a subject, to confirm an additional treatment, and to prepare a report. When the inconsistency is found between an electronic medical record and a clinical report recorded in Electronic Data Capture (EDC), such a cost occurs that is required to provide a hearing to a physician and/or a nurse for example, to correct the data, and to recalculate a statistic value. If any missed test value is found, such a cost occurs that is required to test the subject to correct the data.

FIG. 5 is a diagram for illustrating an example of a configuration of the risk evaluation model 22.

The risk evaluation model 22 includes the record of the risk evaluation result of each execution facility including an incident 221, an average occurrence number 222, and a risk level 223.

The incident 221 shows the type of an incident occurred in a clinical test. The average occurrence number 222 shows the frequency at which the incident occurs (an average value of the occurrence(s) during a unit period). The risk level 223 shows the risk level of the execution facility of the incident handling cost table 21 (e.g., three risk stages of H: high risk, M: medium risk, L: low risk).

Specifically, the risk evaluation model 22 includes the record of the risk level of each execution facility. Different risk levels may be provided to different execution facilities for different incidents.

For example, Nij (“i” shows an incident number and “j” shows a facility number) has an initial value set to any of NiH, NiM, or NiL based on the test result performed on the CRC described later. With the subsequent progress of the clinical test, the initial value set may be updated based on an actual incident occurrence frequency. The reference numeral “Lij” shows the risk level of the incident i of the execution facility j and may be simply defined as a value set to any of H, M, or L depending on the value of “Nij” for example. The value of “Nij” may be updated by any arbitrary method using a Bayes model for example. In this case, the initial value of the average number may be set to the average of the distribution prior to the start of the clinical test for example and the updated value may be set to the average value of the distribution after the start of the clinical test.

FIG. 6 is a diagram for illustrating an example of a configuration of the monitor unit price table 23.

The monitor unit price table 23 includes the record of the cost of the clinical development monitor and includes a monitor 231 and a unit price 232.

The monitor 231 is identification information (e.g., ID, name) to uniquely identify the CRA. The unit price 232 shows the cost per a unit time of the CRA and is recorded as a positive value represented by a currency unit for example.

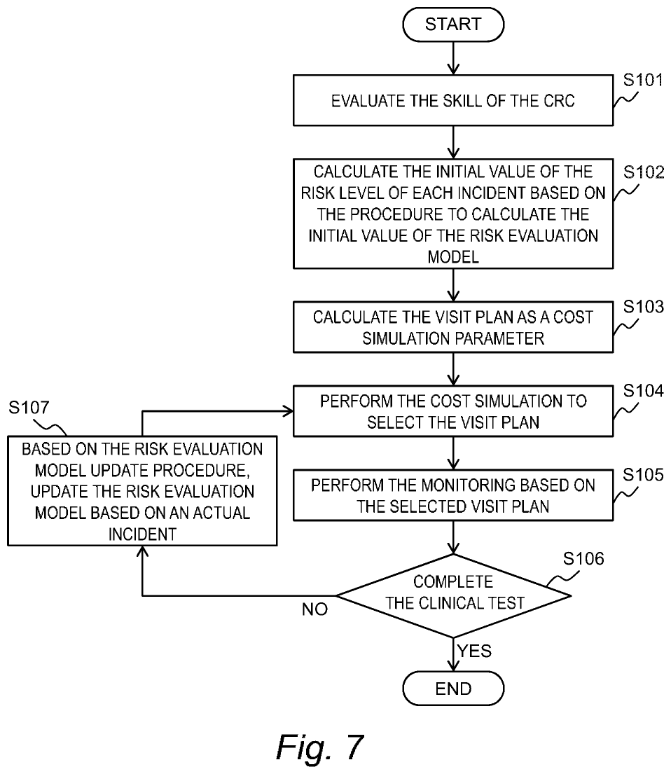

FIG. 7 is a flowchart of a clinical test support procedure.

First, in order to evaluate the skill of the CRC, the processor 101 provides a test problem to the CRC of the execution facility to register the result (score) (S101). Thereafter, the processor 101 launches the risk evaluation model calculation unit 30 to calculate the initial value of the risk level of each incident based on the test result of the CRC and a procedure to calculate the initial value of the risk evaluation model (S102). The details of the procedure to calculate the initial value of the risk evaluation model will be described with reference to FIG. 13.

Next, the processor 101 launches the visit plan preparation unit 10 to prepare many visit plans 15 based on a combination of the CRA, the visit timing, and the execution facility in order to use the combination for the cost prediction by the cost prediction unit 20 (S103).

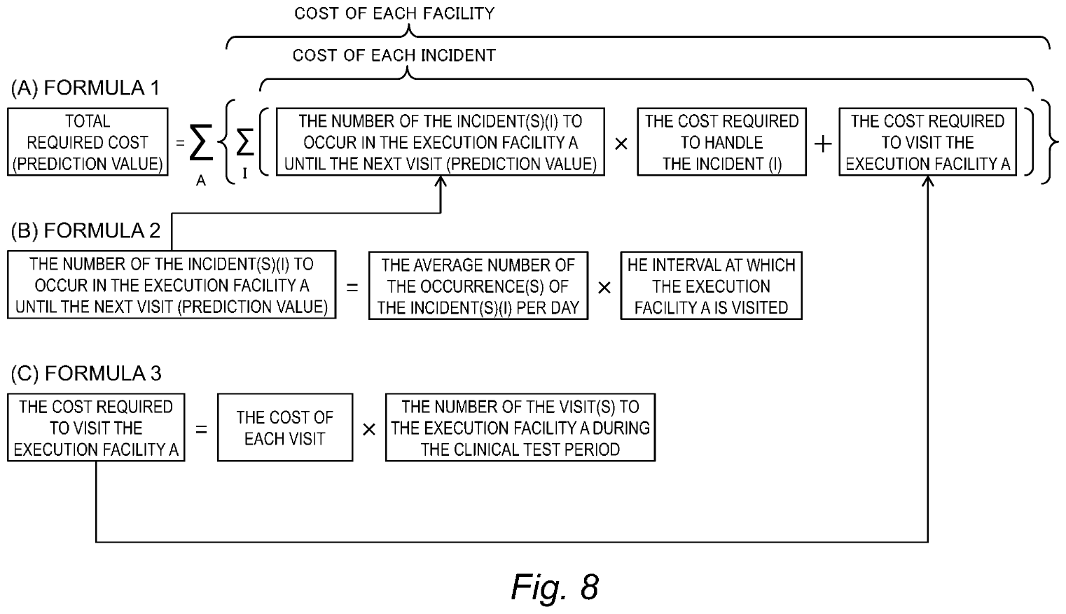

Next, the processor 101 launches the cost prediction unit 20 to execute a cost simulation to calculate the CRA visit cost required when the visit plan 15 prepared by the visit plan preparation unit 10 is carried out. The following section will describe the method to calculate the visit cost with reference to FIG. 8. Based on the calculated visit cost, a visit plan to be suggested to the user is selected (S104). Regarding the number of the visit plan(s) 25, one visit plan 25 providing the lowest cost may be selected or a plurality visit plans 25 requiring a reduced cost may be selected within a selectable range.

In this embodiment, the costs of many visit plans 15 were calculated and a visit plan requiring a low cost was selected. However, another configuration may be used in which a visit plan having an optimal cost is calculated based on one visit plan 15 as a starting point that is subjected to a recursive operation using different parameters (CRAs to visit the facility or the visit timing).

Alternatively, a CRA capacity model may be prepared so that a CRA having a high capacity can visit an execution facility having many incidents in a prioritized manner. Specifically, the unit price of the CRA is adjusted depending on the capacity.

Thereafter, based on the selected visit plan, the clinical test is carried out. The processor 101 receives the input of the monitoring result (S105). An incident inputted during the monitoring is registered in the incident database 33.

Thereafter, after the completion of the clinical test (YES in S106), the clinical test support procedure is completed. When the clinical test is not completed (NO in S106) on the other hand, the risk evaluation model 22 is updated based on an actual incident in accordance with a risk evaluation model update procedure (S107). For example, the average value of the incident occurrence number of the to-be-updated execution facility may be compared with the incident occurrence numbers of the respective risk levels to update the facility to have a risk level close to the occurrence number. Statistic values other than the average value also may be used to determine the risk level of the execution facility. Generally, the incident occurrence number obtained by a Poisson distribution may be used to update the risk level having the largest overlapped distribution. This update of the risk evaluation model 22 may be carried out with a predetermined timing (e.g., a predetermined time period such as every week or a timing at which the visit to all execution facilities is completed).

FIG. 8 is a diagram for illustrating a method of the cost simulation by the cost prediction unit 20.

The formula 1 shown in FIG. 8A is a formula to calculate the total value of the visit cost of the CRC required for one visit plan (the total required cost (prediction value)). The cost of the incident (I) can be calculated based on the following: the number (prediction value) of the incident(s) (I) to occur in the execution facility A until the next visit×the cost to handle the incident(s) (I)+the cost to visit the execution facility A.

The number (prediction value) of the incident(s) (I) to occur in the execution facility A until the next visit can be calculated, as shown in the formula 2 (FIG. 8B), based on the average occurrence number per day of the incident(s) (I)×the interval at which the execution facility A is visited. The average occurrence number per day of the incident(s) (I) is acquired from the risk evaluation model 22. The interval at which the execution facility A is visited is acquired from the visit plan 15 as a cost prediction parameter.

The cost to handle the incident (I) is acquired from the incident handling cost table 21.

The cost required to visit the execution facility A is determined by the person visiting the facility and the number of the visit(s). As shown in the formula 3 (FIG. 8C), this cost can be calculated by the cost of each visit×the number of the visit(s) to the execution facility A during a clinical test period. The cost of each visit is acquired from the monitor unit price table 23. The number of the visit(s) to the execution facility A during the clinical test period is acquired from the visit plan 15 as a cost prediction parameter.

The total value of the visit cost of the CRA for one visit plan can be calculated by calculating the sum of the costs of all incidents for all execution facilities calculated in the above-described manner.

Next, the following section will describe the risk evaluation model calculation unit 30 as an optional configuration of the clinical test support system 1. The clinical test support system 1 may perform the cost simulation using the risk evaluation model 22 given in advance. Alternatively, the risk evaluation model calculation unit 30 may prepare the risk evaluation model 22.

FIG. 9 is a diagram for illustrating a configuration of the periphery of the risk evaluation model calculation unit 30.

The risk evaluation model calculation unit 30 generates the risk evaluation model 22 by referring to a risk evaluation parameter initial value table 32, the incident database 33, and the test evaluation result database 34. The risk evaluation parameter initial value table 32, the incident database 33, and the test evaluation result database 34 are stored in the axillary storage device 103.

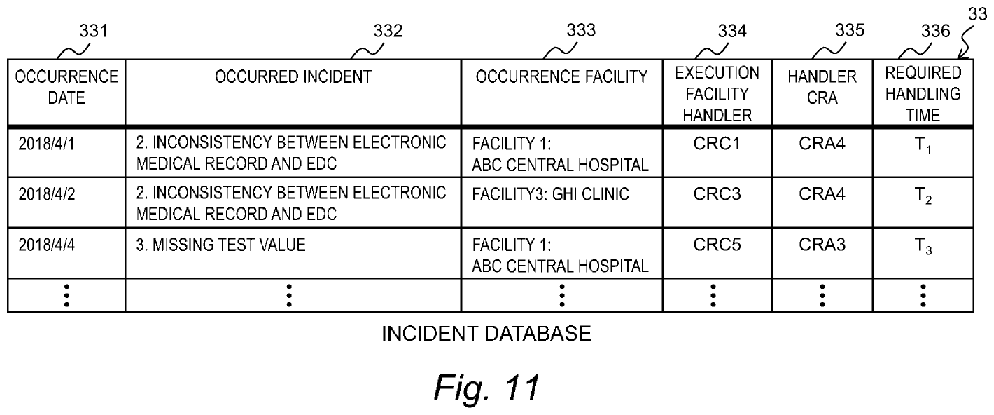

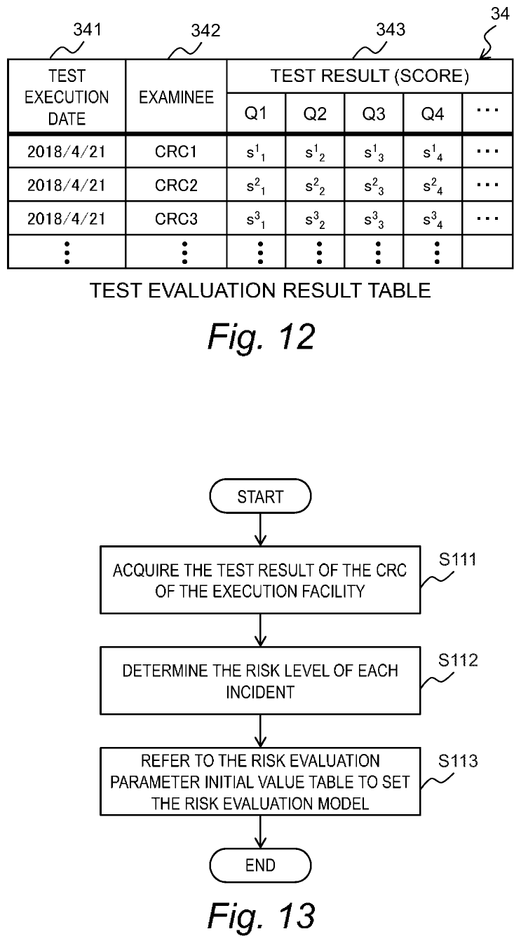

The risk evaluation parameter initial value table 32 includes the number of the occurrence(s) of the incident(s) of the respective risk levels, a configuration example of which is shown in FIG. 10. The incident database 33 includes the record of the incident occurred in the execution facility, a configuration example of which is shown in FIG. 11. The test evaluation result database 34 includes the record of the test result of the CRC, a configuration example of which is shown in FIG. 12.

FIG. 10 is a diagram for illustrating a configuration example of the risk evaluation parameter initial value table 32.

The risk evaluation parameter initial value table 32 includes the record of the number of the occurrence(s) of the incident(s) of the respective risk levels, including incidents 321 and risk levels 322.

The incident 321 shows the type of the incident occurred in the clinical test. The risk level 322 shows the frequency at which the incident occurs for each risk level (the average value of the number of occurrences per a unit period). The risk level 322 recorded in the risk evaluation parameter initial value table 32 can be determined based on the number of the occurrence(s) of the incident(s) recorded in the incident database 33 as described later. Alternatively, the risk level 322 may be determined based on the knowledge owned by specialists.

FIG. 11 is a diagram for illustrating a configuration example of the incident database 33.

The incident database 33 includes the record of the incident(s) occurred in the execution facility, including an occurrence date 331, an occurrence incident 332, an occurrence facility 333, an execution facility handler 334, a handler CRA 335, and the required handling time 336.

The occurrence date 331 shows a date at which the incident occurred. The occurrence incident 332 shows the type of the occurred incident, including, for example, an electronic medical record, the inconsistency with EDC data, or a missing test value. The occurrence facility 333 shows the name of the execution facility in which the incident occurred. Instead of the name of the execution facility, the identification information of the execution facility may be recorded. The execution facility handler 334 shows the identification information of the CRC handling the incident. The handler CRA 335 shows the identification information of the CRA handling the incident. The required handling time 336 shows the time required for the CRA to handle the incident.

FIG. 12 is a diagram for illustrating a configuration example of the test evaluation result database 34.

The test evaluation result database 34 includes the record of the test taken by the CRC, including a test execution date 341, an examinee 342, and a test result 343.

The test execution date 341 shows the test execution date (year, month, and day). The examinee 342 shows the information of the identification of the tested CRC. The test result 343 shows the score of each problem. The problem of the test to be taken by the CRC is related to an important point to reduce the incident occurrence and how to handle the incident occurrence for example. The score of each problem can provide the estimate of the probability at which the incident may occur.

FIG. 13 is a flowchart of a procedure to calculate the risk evaluation model initial value.

First, the risk evaluation model calculation unit 30 acquires the test result of the CRC of the execution facility (e.g., the scores of the respective problems) (S111).

Thereafter, an average mark of the test result of the CRC of the execution facility is calculated. Based on the calculated average mark, the initial value of the risk level of the incident related to the problem is determined as any of the three stages of H: high risk, M: medium risk, and L: low risk, for example (S112).

Thereafter, the number of the occurrence of the incident of each risk level recorded in the risk evaluation parameter initial value table 32 is referred to determine the initial value of the average occurrence number of each incident based on the risk level determined based on the test result of the CRC.

Then, the initial value of the average occurrence number of the incident and the initial value of the risk level are recorded in the risk evaluation model 22 to determine the initial value of the risk evaluation model 22 (S113).

In this embodiment, the cost was described as an example of the evaluation index. However, various evaluation indexes as shown below also can be used by the calculation by the cost prediction unit 20.

1. The number of responsible CRA(s): The number of the responsible CRA(s) is calculated by counting the number of the CRA(s) unique to the visit plan. This allows a visit plan requiring less CRA(s) to be selected.
2. Levelling of working hours of the CRA: The visit numbers of the respective CRAs in the visit plans are counted to calculate the dispersion. This provides the selection of a lower dispersion value, thereby providing the selection of a visit plan for which the working hours are leveled.
3. Low risk: A low-risk visit plan can be selected by selecting a visit plan for which “the cost to visit the execution facility A” is 0 in the formula 1 of FIG. 8A based on a cost to handle the incident occurrence. This consequently provides, by selecting a visit plan having a lower value in the formula, the selection of a low-risk visit plan.

As described above, according to the clinical test support system 1 of this embodiment, the cost prediction unit 20 calculates the evaluation index used to perform the visit plan 15 prepared by the visit plan preparation unit 10 by referring to the incident handling cost table 21 (evaluation index information) and the risk evaluation model 22. Then, the clinical test support system 1 outputs a visit plan selected based on the evaluation index. Thus, the visit plan of the CRA can be evaluated depending on the management index, thus providing the selection of the visit plan matching the evaluation index.

The cost prediction unit 20 calculates the cost to perform the visit plan 15 prepared by the visit plan preparation unit 10 to output the visit plan 25 requiring a low cost by referring to the incident handling cost table 21 (evaluation index information), the risk evaluation model 22, and the monitor unit price table 23 (monitor unit price information) to. Thus, the visit cost of the CRA required to perform the visit plan can be calculated, thus providing the selection of a low-cost visit plan.

Furthermore, the cost prediction unit 20 can calculate the cost to perform the visit plan by calculating the sum of a value obtained by multiplying the number of at least one of the occurrence(s) of at least one of the incident(s) during the clinical test period with the cost required to handle the incident and the cost required to visit the execution facility. Thus, the cost can be quickly calculated by a simple four arithmetic operation, thus providing the selection of a visit plan having an optimal cost from among many visit plans 15.

Furthermore, the risks of the respective execution facilities can be estimated by the risk evaluation unit (the risk evaluation model calculation unit 30) that predicts the amount of at least one of the occurrence(s) of at least one of the incident(s) in the execution facility to calculate the risk evaluation model 22 based on the test evaluation result database 34 (evaluation result information) and the incident database 33 (incident information).

Furthermore, the risk evaluation model calculation unit 30 determines the risk level of the execution facility based on the evaluation result regarding the clinical test of the CRC for the execution facility recorded in the test evaluation result database 34. Thus, the risk level can be set depending on the actual capability of the CRC belonging to the execution facility.

Furthermore, the risk evaluation model calculation unit 30 calculates the average value of the at least one of occurrence(s) of at least one of the incident(s) recorded in the incident database 33 as the risk evaluation model 22 based on the risk level of the execution facility, thus providing the prediction of the number of the occurrence(s) of the incident(s) matching the risk level of the execution facility.

Furthermore, the risk evaluation model calculation unit 30 determines, after the start of the clinical test and after the update of the incident information, the risk level of the execution facility based on the number of at least one of the occurrence(s) of at least one of the incident(s) of the execution facility, thus setting the appropriate risk level based on the latest information.

This invention is not limited to the above-described embodiments but includes various modifications. The above-described embodiments are explained in details for better understanding of this invention and are not limited to those including all the configurations described above. A part of the configuration of one embodiment may be replaced with that of another embodiment; the configuration of one embodiment may be incorporated to the configuration of another embodiment. A part of the configuration of each embodiment may be added, deleted, or replaced by that of a different configuration.

The above-described configurations, functions, processing modules, and processing means, for all or a part of them, may be implemented by hardware: for example, by designing an integrated circuit, and may be implemented by software, which means that a processor interprets and executes programs providing the functions.

The information of programs, tables, and files to implement the functions may be stored in a storage device such as a memory, a hard disk drive, or an SSD (a Solid State Drive), or a storage medium such as an IC card, or an SD card.

The drawings illustrate control lines and information lines as considered necessary for explanation but do not illustrate all control lines or information lines in the products. It can be considered that almost of all components are actually interconnected.

1. A clinical test support system, comprising:
an arithmetic unit for performing a predetermined processing; and
a storage device coupled to the arithmetic unit,
a visit plan preparation unit in which the arithmetic unit is configured to prepare a visit plan for the execution facility of a clinical development monitor; and
a prediction unit in which the arithmetic unit is configured to calculate the evaluation index of the visit plan, wherein
the storage device stores evaluation index information including the record of information for the evaluation of the visit plan and a risk evaluation model including the record of the risk evaluation result of each execution facility,
the prediction unit is configured to:
calculate an evaluation index used to execute the visit plan prepared by the visit plan preparation unit by referring to the evaluation index information and the risk evaluation model; and
output a visit plan selected based on the evaluation index.

2. A clinical test support system according to claim 1, wherein
the prediction unit is configured to calculate the cost to perform the visit plan as an evaluation index,
the evaluation index information includes the record of the cost required to handle each incident in a clinical test as the information for the evaluation of the visit plan,
the storage device stores monitor unit price information including information for the cost of the clinical development monitor, and
the prediction unit is configured to:
calculate the cost required to execute the visit plan prepared by the visit plan preparation unit by referring to the evaluation index information, the risk evaluation model, and the monitor unit price information; and
output a low-cost visit plan.

3. A clinical test support system according to claim 2, wherein
the prediction unit is configured to calculate the cost to perform the visit plan by calculating the sum of a value obtained by multiplying the number of at least one of the occurrence of at least one of the incident during a clinical test period with the cost required to handle the incident and the cost required to visit the execution facility.

4. A clinical test support system according to claim 1, wherein
the storage device stores incident information including the record of the incident occurred in the past clinical test and evaluation result information including the record of information regarding the level of the handling by the execution facility at the occurrence of incident, and
the clinical test support system comprises a risk evaluation unit in which the arithmetic unit is configured to predicts the amount of at least one of the occurrence of the incident in the execution facility to calculate the risk evaluation model based on the evaluation result information and the incident information.

5. A clinical test support system according to claim 4, wherein
the incident information includes the correspondence information between an execution facility and a coordinator, and
the risk evaluation unit is configured to determine the risk level of the execution facility based on the evaluation result regarding a clinical test of the coordinator in the execution facility recorded in the evaluation result information.

6. A clinical test support system according to claim 5, wherein
the risk evaluation unit is configured to calculate the statistic value of the number of at least one of the occurrence of at least one of the incident recorded in the incident information as the risk evaluation model based on the risk level of the execution facility.

7. A clinical test support system according to claim 4, wherein
the risk evaluation unit determines, after the start of the clinical test and after the update of the incident information, the risk level of the execution facility based on the number of at least one of the occurrence of at least one of the incident of each execution facility.

8. A non-transitory machine-readable storage medium, containing at least one sequence of instructions for performing a clinical test support method by a computer, wherein:
the computer has an arithmetic unit for performing a predetermined processing and a storage device coupled to the arithmetic unit, and
the storage device stores evaluation index information including the record of information for the evaluation of the visit plan and a risk evaluation model including the record of the risk evaluation result of each execution facility,
the instructions that, when executed, cause the computer to:
prepare a visit plan for an execution facility of a clinical development monitor;
calculate an evaluation index used to perform the visit plan prepared by the visit plan preparation step by referring to the evaluation index information and the risk evaluation model; and
output the visit plan selected based on the evaluation index.

9. The non-transitory machine-readable storage medium according to claim 8, wherein
the arithmetic unit is configured to calculate, as the evaluation index, the cost to perform the visit plan,
the evaluation index information includes the record of the cost required to handle each incident in a clinical test as the information for the evaluation of the visit plan,
the storage device stores monitor unit price information including information for the cost of the clinical development monitor,
wherein the instructions further cause the computer to:
calculate the cost to perform the visit plan prepared by the visit plan preparation step by referring to the evaluation index information, the risk evaluation model, and the monitor unit price information; and
output the visit plan having a low cost.

10. The non-transitory machine-readable storage medium according to claim 9, wherein
the instructions further cause the computer to calculate the cost to perform the visit plan based on the sum of a value obtained by multiplying the number of at least one of the occurrence of at least one of the incident during a clinical test period with the cost required to handle the incident and the cost required to visit the execution facility.

11. The non-transitory machine-readable storage medium according to claim 8, wherein
the storage device stores incident information including the record of the incident occurred in the past clinical test and evaluation result information including the record of information regarding the level(s) of the handling by the execution facility at the occurrence of incident(s), and
the instructions further cause the computer to perform a risk evaluation step to predict the amount of at least one of the occurrence of the incident in the execution facility to calculate the risk evaluation model based on the evaluation result information and the incident information.

12. The non-transitory machine-readable storage medium according to claim 11, wherein
the incident information includes the correspondence information between an execution facility and a coordinator, and
the instructions further cause the computer to determine the risk level of the execution facility based on the evaluation result regarding a clinical test of a coordinator in an execution facility recorded in the evaluation result information.

13. The non-transitory machine-readable storage medium according to claim 12, wherein
the instructions further cause the computer to calculate the statistic value of the number of at least one of the occurrence of at least one of the incident recorded in the incident information as the risk evaluation model based on the risk level of the execution facility.

14. The non-transitory machine-readable storage medium according to claim 11, wherein
the instructions further cause the computer, after the start of the clinical test and after the update of the incident information, to calculate the risk level of the execution facility based on the number of at least one of the occurrence of at least one of the incident of each execution facility.

15. A clinical test support method performed by a computer, wherein
the computer has an arithmetic unit for performing a predetermined processing and a storage device coupled to the arithmetic unit, and
the storage device stores evaluation index information including the record of information for the evaluation of the visit plan and a risk evaluation model including the record of the risk evaluation result of each execution facility,
the clinical test support method includes:
a visit plan preparation step to prepare a visit plan for an execution facility of a clinical development monitor;
a cost prediction step to calculate an evaluation index used to perform the visit plan prepared by the visit plan preparation step by referring to the evaluation index information and the risk evaluation model; and
an output step to output the visit plan selected based on the evaluation index.
